(19) United States
(12) Patent Application Publication
(10) Pub. No.: US 2006/0229564 A1
(43) Pub. Date: Oct. 12, 2006

(54) Closure with passage slit for a haemostatic valve assembly

(76) Inventors: Erik Andersen, Roskilde (DK); Finn Birk Pedersen, Stenlose (DK)

Correspondence Address: BIRCH STEWART KOLASCH & BIRCH, PO BOX 747, FALLS CHURCH, VA, 22040-0747, US

(21) Appl. No.: 10/560,827

(22) PCT Filed: Jun. 17, 2004
(86) PCT No.: PCT/DK04/00424

§ 371 Date: May 19, 2006

Related U.S. Application Data
(60) Provisional application No. 60/478,832, filed on Jun. 17, 2003.
(60) Provisional application No. 60/496,910, filed on Aug. 22, 2003.

(30) Foreign Application Priority Data
Jun. 17, 2003 (DK) PA 2003 00896
Aug. 22, 2003 (DK) PA 2003 01205

(51) Int. Cl. A61M 5/178; A61M 39/10; A61M 39/00; A61M 25/16; A61M 25/18

(52) U.S. Cl. 604/167.03; 604/533

(57) ABSTRACT

A closure for a valve (128) of a connector (100) of a haemostatic valve assembly comprises a resilient closure member (130), which defines a first and a second, opposite end surface (156; 160) and at least one passage slit (164). The passage slit is normally closed and extends between the two end surfaces, the passage slit being arranged to open by a tubular member (134) being extended therethrough, the passage slit (164) having a wider extent at the first surface than at the second surface. A plurality of such passage slits (164) may define a first, common point of contact (166) on the first surface (156) and may extend radially outwardly from the point of contact (166) at the first surface (156).

### TECHNICAL FIELD

The present invention generally relates to the field of connectors for haemostatic valve assemblies, as used for example in angioplasty. An elongate member, such as a balloon catheter or a vascular stent, may be introduced into the vascular system of a living being through the connector which incorporates a haemostatic valve for safe haemostasis. In particular, the present invention provides an improved closure member for haemostatic valve assemblies. More specifically the present invention relates to a closure made from a resilient material and including a slit which extends between two opposed surfaces.

### BACKGROUND OF THE INVENTION

Access to the vascular system of a living being, such as a cardiac patient, is required during endovascular procedures such as in angioplasty, e.g., for the introduction of balloon catheters or stent systems. Usually, access is provided via a connector which, e.g., provides a connection to a guiding catheter, the connector integrating a haemostatic valve to enable an elongate device to be introduced into the body of the living being while providing safe haemostasis. A side arm may be provided as a part of such a connector in order to provide a connection to a manifold used for pressure monitoring, contrast media injection and/or saline flushing. Connectors with side arms are normally referred to as ‘Y-connectors’. The haemostatic valve ensures that blood does not flow out of the connector while enabling a catheter, stent system or arterirectomy device to be passed through the connector. At the distal end of the connector there may be provided a rotatable luer for securing the connector to a corresponding member at the proximal end of a guide catheter.

U.S. Pat. No. 5,195,980 (David G. Catlin), discloses a haemostatic valve comprised in a Y-connector. The haemostatic valve is incorporated in a proximal end of a main section of the connector, which comprises a rotatable luer at its distal end. A side arm joins the main section between the distal end and the haemostatic valve. There is further disclosed a resilient valve element including a normally closed slit which is arranged to be opened by an access tube being extended therethrough. Another example of a haemostatic valve is known from U.S. Pat. No. 5,176,652 (Perry K. Littrell), the haemostatic valve of U.S. '652 including two elastic and gaskets having slits capable of permitting an elongated member to extend therethrough, with the slits extending completely through the respective gaskets and the gaskets being angularly displaced with respect to one another. U.S. '652 and U.S. Pat. No. 4,798,594 (Richard A. Hilistead) further disclose helically extending slits.

The art of coronary angioplasty is generally described in: Coronary Angioplasty by Bernhard Meier, published by Grune & Stratton, Inc., Harcourt Brace Jovanovich, Publishers, 1987.

### SUMMARY OF THE INVENTION

The present invention alms at providing an efficient sealing between a passage in the connector and a surrounding atmosphere, in particular a sealing at a proximal end of the connector while allowing for easy introduction of a catheter or stent system through the closure. The closure of the invention is made from a resilient material and includes a slit which extends between two opposed surfaces. The slit is configured to provide a closure with reliable sealing when a catheter or stent system extends through the closure and further to provide a reliable closure when the valve is in a closed state, i.e. when no catheter or stent system extends through the closure.

Accordingly, the invention provides a closure for a valve of a connector of a haemostatic valve assembly, the closure comprising a closure member which is made from a resilient material and which defines a first and a second end surface, which is opposed to the first end surface, and at least one passage slit, the passage slit being normally closed and extending between the two end surfaces, the passage slit being arranged to open by a tubular member being extended therethrough, the passage slit having a length at the first surface which is longer than its length on the second surface, i.e. the passage slit having a wider extent at the first surface than at the second surface. In other words, at one surface of the closure member, the transverse length of the slit is shorter than the transverse length of the slit at the other surface of the closure member. Thereby, the slit defines a guide for the catheter or stent system when such a system is moved through the closure, the guide at least partially forcing the member being introduced through the closure into a particular angular alignment with respect to the closure. In a preferred embodiment, the closure is a gasket-like member made from a resilient or elastomeric material, such as silicone or latex. The passage slit is preferably formed such that it defines a first axis of symmetry on the first surface which is aligned with a second axis of symmetry on the second surface, such that the catheter or stent system is held substantially perpendicular to the end surfaces of the closure when extending therethrough. By providing a slit as explained above, the risk of improper alignment of the catheter or stent system and the closure is reduced and thereby also the risk of the catheter or stent system stretching/deforming the closure to such an extent in the area of the slit that a gap is created between an outer wall of the catheter or stent system and the closure.

In preferred embodiments of the invention, there are provided a plurality of passage slits, which define a first, common point of contact or point of intersection on the first surface, and which extend radially outwardly from the point of contact at the first surface. There may for example be provided three silts which extend radially from the point of contact, or there may be provided four slits which are arranged to form a cross. On the second, opposed surface, the slits preferably define a second, common point of contact, the slits preferably being substantially short at the second surface than at the first surface. Preferably, the length of each slit at the second surface is at most 1/10th of the length of that slit at the first surface, more preferably at most 1/20th. In an idealistic embodiment, the slits extend a length close to zero at the second surface, such that they meet in a point on the second surface, the point being preferably arranged centrally with respect to the surface.

In order to facilitate introduction of a catheter or stent system through the closure, at least a portion of one of the first and second end surfaces, such as preferably the second end surface, may have a concave shape.

When mounted in a connector, the second end surface is preferably oriented to face the proximal end of the connector.

The invention further aims at providing an efficient sealing between a passage in the connector and a surrounding atmosphere, in particular a sealing at a proximal end of the connector while allowing for easy introduction of a catheter or stent system through the closure. Accordingly, a face of the closure member may abut a proximal end surface of a main section of the connector. It is desired to provide a closure which includes a reliable seal at a periphery of the main section of the connector. The connector may comprise a longitudinally extending main section having a longitudinally extending, through-going passage with the valve at a proximal end of the connector. The closure may comprise a closure member, a face of which abuts a proximal end surface of the main section, one of said face and end surface being provided with a protrusion for engaging a corresponding indentation provided in the other one of the face/surface. It will be appreciated that the protrusion and indentation provide a further sealing in comparison to the sealing provided by traditional, planar gaskets. In a preferred embodiment, the closure member is made from a resilient material which is adapted to deform in the area of the protrusion/indentation when the face of the resilient closure and the end surface of the main section are biased towards each other. Thereby, a liquid tight seal is provided at the outer periphery of the passage at a proximal end thereof. Preferably, the protrusion and indentation extend over an angle of 360°, so that the seal is efficient along the entire end surface of the main section. The protrusion and indentation preferably extend along a peripheral section of, e.g., the closure member. The protrusion, which may be formed as an integral part of the closure member, may extend along a peripheral section of that surface which faces an end surface of the connector. Preferably, the protrusion extends in a longitudinal direction, i.e. transverse to the plane of its end surface. The protrusion may be tapered, so that it is wider at its proximal end than at its distal end. The closure member may define a core section which fits into a longitudinal passage in the connector. The core section may be tapered, so that its diameter is larger at its proximal end than at its distal end.

As mentioned previously, the closure may be incorporated in a connector comprising a longitudinally extending main section with a valve at a proximal end thereof, the valve having an open state in which an elongate member may be inserted into the passage, and a closed state. Before introducing a device, e.g., a catheter or a drug-coated stent, through the connector and into the vascular system of a living, an operator, such as a physician, should ensure that the valve is properly opened, as otherwise an outer surface of the catheter or stent risks to scrape against parts of the valve, with the result that the surface of the device is damaged or that accurately dosed drug provided on the surface of a drug-coated stent is lost. However, given exterior circumstances such as in particular psychological stress, an operator may sometimes not verify that the valve is in its open state before attempting to introduce the device through the valve. Following an attempt to introduce the device through a closed valve, the operator may not always realise that drug has been scraped off the stent or that physical damaged has been caused to a surface of the device, and he may, after having properly opened the valve, introduce the device, now, for example, damaged or with a wrong dose of drug on the surface thereof, into the vascular of the patient. Such an incidence may seriously compromise the patient's health and does often result in the need for additional treatment and prolonged hospitalisation of the patient. Accordingly, it is desired to provide a means for reducing the risk of causing damage to a device to be inserted into the vascular system through the valve of a connector. Thus, the valve, which is preferably arranged at a proximal end of the connector, may have an open state in which an elongate member may be inserted into the passage, and a closed state, the valve comprising an indicator for indicating the state of the valve. The indicator may provide an optical and/or a tactile feedback to an operator, so that the operator by looking at or by touching the valve may easily determine the state of the valve.

In a preferred embodiment, the valve includes a valve opener which is longitudinally displaceable along an outer surface of the main section of the connector, such that the state of the valve may be changed by displacing the valve opener in relation to the main section. The valve opener, or, in case of other embodiments, other displaceable means, may advantageously be arranged near the indicator which may comprise optical means for providing an optical appearance of at least a part of the connector in the open state which is different from, an optical appearance of that part of the connector in the closed state. For example, the valve may comprise an elastomeric closure member, such as a silicone member, arranged to seal the proximal end of the connector in the closed state of the valve, the valve opener comprising a puncture member which extends co-axially with and at least partly inside said passage. The puncture member may be arranged such with respect to the closure member that it penetrates the closure member in the open state of the valve, the elastomeric closure member thereby closing about an outer surface of the puncture member, and such that it does not penetrate the closure member in the closed state of the valve. Such an embodiment of the valve is well suited for an embodiment of the valve opener which comprises a transparent portion and an opaque portion, and wherein the main section of the connector, at a proximal end thereof, comprises a coloured section which is covered by the opaque portion of the valve opener when the valve is in the open state, and which is visible through the transparent section when the valve is in the closed state. Thus, for example the coloured section may be clearly visible to the operator when the valve is in the closed state and completely hidden when the valve is in the open state. Accordingly, a superficial and rapid glance at the valve may allow the operator to determine the state of the valve. Preferably, a proximal end surface of the valve opener is opaque, so that an operator does not risk to see the coloured section through the proximal end surface in that state of the valve, in which the coloured section should be hidden.

Though not preferred, a so-called ‘Touhy Borst’ valve, which is known per se, and which comprises an elastomeric membrane having an opening through which the catheter extends and which is closed about the periphery of the catheter by rotation of a cap, may be provided as the haemostatic valve. However, from an ease-of-use point of view, the ‘Touhy Borst’ design has the disadvantage that it requires a separate introducer needle or tube to pass thorough the valve for opening the membrane, so that a catheter or stent can be introduced without damage. Therefore, as the introduction and common use of vascular stents, including balloon expandable stents and self-expanding stents, has resulted in increased attention to the friction in passing a device through the valve and to the need for maintaining a position of the stent on the balloon, so-called puncture valves have become more popular. Examples of such puncture valves are those described herein in connection with the preferred embodiments of the present invention, the valve disclosed in U.S. Pat. No. 5,195,980, and the valve described in U.S. Pat. No. 5,176,652.

Embodiments of the connector comprising a side arm for connecting the connector to a manifold, i.e. so-called ‘Y-connecter’ embodiments, may, according to the invention, be comprised in a kit further comprising a side arm tubing for the side arm and possibly a stopcock.

Certain embodiments of the connector of the invention may have a main section being manufactured from two separate, co-extending parts which are mutually interconnected or joined, the two parts being preferably made from a plastics material. When interconnected, the two parts should be able to withstand a certain pressure in a longitudinal passage extending inside and being defined by inner surfaces of the two parts, such as an injection pressure. It has been found that it is sometimes difficult to manufacture an essentially glued interconnection between such separate parts of a connector, as it may not be easy to accurately control the manufacturing process such that the completed connector with certainty will be able to withstand a certain pressure. It is therefore desired to provide a connector for a haemostatic valve assembly comprising two separate parts, which connector does not rely on glue as the single or main means of interconnection of the two parts, while ensuring a relatively uncomplicated and cost efficient manufacturing process.

The longitudinally extending main section of the connector may be manufactured from a proximal part and a distal part. Each of the distal and proximal parts of the main section may define a longitudinally extending, through-going passage. The connector may further comprise connection means for providing a connection between the proximal part and the distal part, whereby, when interconnected, the distal and proximal parts coextend in the longitudinal direction, the connection means comprising a projecting portion which is integral with one of said parts and which is adapted to engage a recessed portion of the other one of said parts, so as to mutually secure the parts in the longitudinal direction. Thus, thanks to the essentially mechanical connection between the proximal and the distal part, the connector may be designed to withstand a given internal pressure, which may accurately be calculated based on specifications of the materials from which the two parts are made and on dimensions of the parts. In addition to the mechanical means provided at the interconnection, the interconnection may be reinforced by glue, though, in a presently preferred embodiment, the connector is assembled without glue. Preferably, the interconnection is formed as a snap-lock connection, e.g. a self securing snap-lock. In a preferred embodiment of the invention, the projecting portion is a barbed portion. The barbed portion may, for example, be provided as a part of an outer periphery of a first one of the two parts, the dimensions of which allows it to at least partially surround an end portion of a second one of the two parts. The surrounded part may thus provide a rim or a collar, e.g., at a transition between a small diameter section and a large diameter section thereof, which rim or collar the barbed portion may engage. The barbed portion preferably includes several barbs arranged along the periphery of the first part. In order to allow the barbed portion of the first part to be slipped over the second part, the barbed portion may be flexible in a radial direction, whereas it is preferred that it is not, or at least less, flexible in the longitudinal direction. Such radial flexibility may be brought about by longitudinally extending slits provided in an end portion of the first part. In one embodiment of the invention, an end portion of the distal part is adapted to receive an end portion of the proximal part, the barbed portion being provided at the proximal end portion of the distal part, the recessed portion comprising a collar portion, e.g. a sharp edged collar portion, provided on an outer surface of the proximal part. In other embodiments, the barbed portion may be provided at an end portion of the proximal part, which may receive an end portion of the distal part.

In order to preclude blood and/or other liquids from flowing out of the connector at the interconnection, there may be provided sealing means at the interconnection. Such sealing means may include a resilient member, such as an O-ring, which, when the proximal and distal parts are interconnected, is clamped between the two parts, for example such that it fits around and tightly closes an outer collar portion of an inner one of the two parts and such that it fits inside and tightly closes an inner collar portion of an outer one of the two parts.

The interconnection may be such that the distal part and the proximal part may rotate relative to each other around an axis extending in the longitudinal direction, such rotation being desired, e.g. If one or both of the two parts are provided with a threaded portion for engaging a thread of a corresponding member, such as of a guide catheter. In a preferred embodiment of the invention, the distal part constitutes a rotatable luer, so that there is no need for manufacturing a luer as a separate part. A first threaded or grooved portion may be provided on an outer and possibly conical wall of the distal part, whereas a second threaded portion may be provided on an inner surface of the distal portion. In the latter case, the threaded portion may be provided between an annular wall surrounding the longitudinally extending passage through the connector and a surrounding outer wall of the distal part, when seen in a radial direction.

The connector may be a Y-connector having a side arm, so as to provide a connection to a manifold used for pressure monitoring, contrast media injection and/or saline flushing. The side arm may be arranged to receive a tube which interconnects the side arm and a stopcock, such as a standard 3-way stopcock. The distal as well as the proximal parts of the connector may be manufactured by injection-moulding of a plastics material.

Generally, embodiments of the connectors of the present invention may be designed to fit a wide variety of stents, including, but not limited to, Strecker Stents, Palmaz Stents, Wallstents, self-expanding Nitinol Stents, such as Bard Luminex Stents, Symphony Stents, Smart Stents and AVE SE Stents, Perflex Stents, AVE Stents, Intrastents, Instents, Herculink, and Dynalink. Likewise, the connectors of the present invention may be designed to fit a variety of catheters, including, but not limited to, Mainz balloon catheters, Monorail balloon catheters, PCTA catheters, and ultrasound catheters.

### BRIEF DESCRIPTION OF THE DRAWINGS

The above aspects of the invention will now be further described with reference to the drawings, in which:

FIG. 1 shows a longitudinal cross-section of a connector according to the invention,

FIG. 2 shows an exploded side view of the connector of FIG. 1 and an associated stopcock,

FIG. 3 shows a longitudinal cross-section of a valve incorporated in the connector of FIGS. 1 and 2, the valve being in a closed state,

FIG. 4 shows the valve of FIG. 3 in an open state,

FIG. 5 shows a main section of a connector with an indentation in an end surface thereof for providing a peripherally extending seal at a proximal end of the connector,

FIG. 6 shows an end view of a closure member for mounting at the proximal end of the connector of FIG. 5,

FIG. 7 is a cross-sectional view of one embodiment of the closure member of FIG. 6,

FIG. 8 is a cross-sectional view of an alternative embodiment of the closure member of FIG. 6,

FIG. 9 shows a cross-section of the valve of FIGS. 3 and 4, including an indicator for indicating a state of the valve,

FIG. 10 shows a longitudinal cross-section of a coloured member comprised in the indicator of the valve of FIG. 9,

FIG. 11 shows a side view of a second embodiment of a connector according to the invention;

FIG. 12 shows a side view of a third embodiment of a connector according to the invention;

FIG. 13 shows a valve opener of the embodiments of FIGS. 11 and 12;

FIG. 14 shows a longitudinal cross-section of an interconnection between a proximal and a distal part of the connector of FIGS. 1, 2, 11 and 12;

FIG. 15 shows a longitudinal cross-section of a distal part of FIG. 14,

FIG. 16 shows a perspective view of the proximal part of FIG. 15.

### DETAILED DESCRIPTION OF THE DRAWINGS

As it will be appreciated from the below description of a preferred embodiment of the invention, all of the above aspects of the invention may be comprised in a single embodiment.

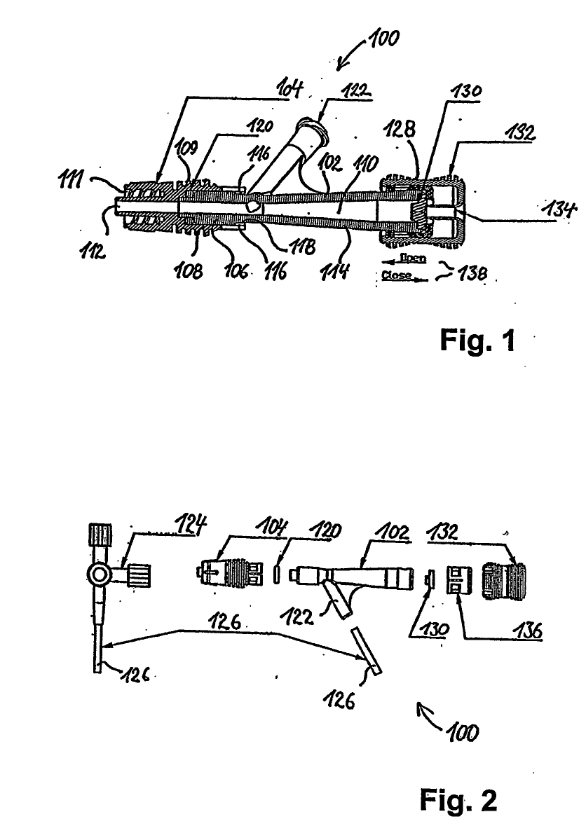

A Y-connector 100, as shown in FIGS. 1 and 2, comprises a proximal part 102 and a distal part 104, the proximal and the distal part co-extending in a longitudinal direction and being assembled in an end-to-end manner with a distal end portion 106 of the proximal part 102 being received in a proximal end portion 108 of the distal part 104. The distal part 104 is formed as a rotating luerlock with a first outer grooved or threaded portion 109, and a second, inner threaded portion 111. Within each of the proximal and the distal part, there is provided a longitudinally extending, through-going passage 110 and 112, respectively. When assembled, the proximal and the distal part together define a main section 114. At the interconnection between the proximal and the distal part, the distal part defines a projecting portion 116, preferably a barbed portion, which projects radially inwardly and engages a recessed portion 118 of the proximal part 102, the recessed portion 118 being in the form of a collar defined by a transition of the outer diameter of the proximal part 102. The interconnection will be further described below with reference to FIGS. 7-9. The projecting portion 116 is shaped to provide a snap-locking of the distal part 104 onto the proximal part 102. When assembled, the proximal part and the distal part clamp an O-ring 120 between them, the O-ring being provided at a reduced-diameter section of the proximal part and at a corresponding widened-diameter section of an inner surface of the distal part. Side arm 122 is provided for connecting the connector 100 to a manifold (not shown) used for pressure monitoring, contrast media injection and/or saline flushing. As shown in FIG. 2, the connection from side arm 122 to the manifold may be provided via a stopcock 124 and a side arm tubing 126.

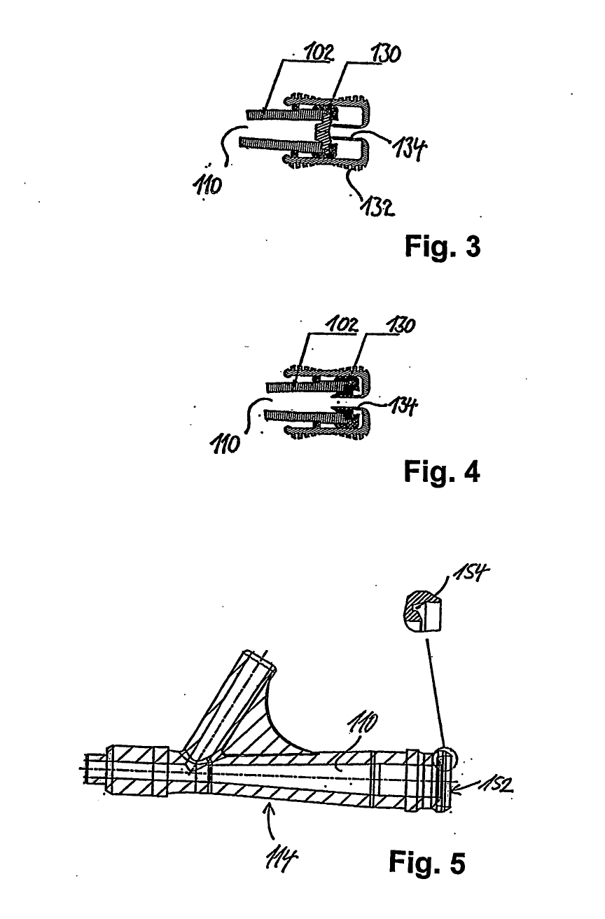

At its proximal end, the connector 100 of FIG. 1 comprises a valve 128 comprising an elastomeric closure member 130, a valve opener 132, and a puncture member 134 shaped to provide an elongate passage port which, in the closed state of the valve as depicted in FIG. 3, allows the closure member to seal the proximal end of the passage 110, whereas in the open state of the valve, as depicted in FIG. 4, the puncture member penetrates the elastomeric closure member 130 to allow a catheter or a stent (not shown) to pass through the valve. The puncture member 134 is, as shown in FIGS. 3 and 4 integral with the valve opener 132, which may be longitudinally displaced along an outer surface of the proximal part 102, as indicated by arrows 138 in FIG. 1, so that in a most proximal position of the valve opener, the valve is in a closed state, as in FIG. 3, and in a most distal position of the valve opener, the valve is in an open state, as in FIG. 4. An indicator for indicating the state of the valve comprises a coloured member 136, see FIG. 2.

Embodiments of the invention will now be further described with reference to FIGS. 5-8, in which elements discussed elsewhere in the present specification are referred to by the same reference numerals to the extent that like parts are referred to. It should, however, be understood that structural differences may exist, despite of the fact that the same reference numerals are employed.

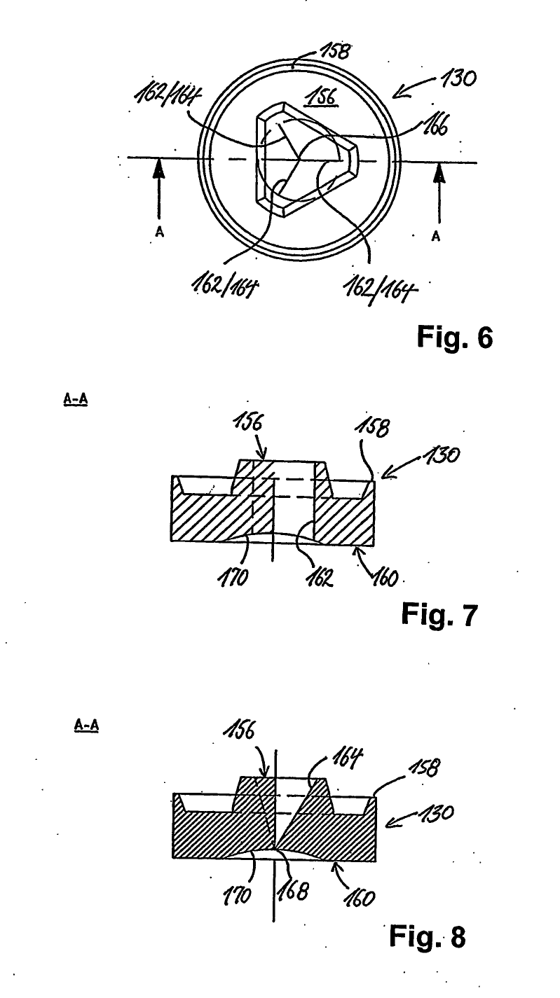

The main section 114 shown in FIG. 5 has a proximal end surface 152 defining an indentation in the form of a groove 154. The groove is intended to receive an annular protrusion 158 provided on the closure member 130, se FIGS. 6-8. When the closure member 130 is biased towards the surface 152 of the main section 114, the protrusion 158 deforms in the groove 154, so that a reliable seal is provided along the end surface 152. The closure 130 is provided with three slits which are referred to by reference numeral 162 in the embodiment of FIG. 7 and-reference numeral 164 in the embodiment of FIG. 8. The slits 162,164 extend radially outwardly from a first, common point of contact 166 provided at a first surface 156 of the closure member 130. In the embodiment of FIG. 7, the slits 162 have a length at the first surface 156 which is substantially equal to their length at a second surface 160 of the closure member. In the embodiment of FIG. 8, the slits 164, however, have a length at the second surface 160 which is close to zero, i.e. the three slits 164 meet in a single, second point of contact 168. In both embodiments, a concave portion 170 is provided to facilitate insertion of a catheter or stent system through the closure member.

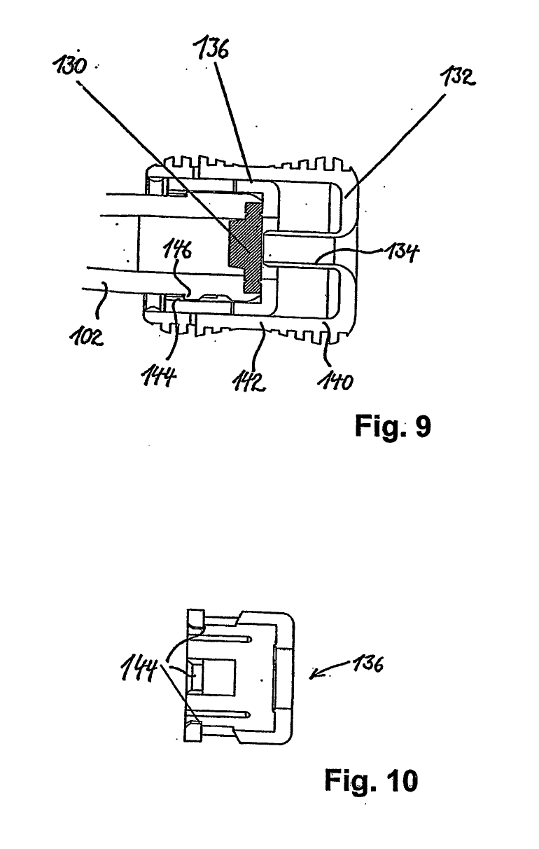

FIG. 9 shows a cross-section of the valve of FIGS. 3 and 4, including an indicator for indicating a state of the valve. The indicator comprises a coloured member 136 which may, e.g., the colour of which may, e.g., be yellow or any other strong colour. A most proximal section 140 of the valve opener 132 is opaque, whereas a distal portion 142 of the valve opener is transparent. Hence, when the valve is in a closed state as illustrated in FIG. 9, the coloured member 136 is visible through the transparent portion 142. However, when the valve is in an open state, i.e. when the valve opener 132 is displaced to the position illustrated in FIG. 4, the opaque section 140 of the valve opener overlaps the coloured member 136, which is then essentially invisible to an operator. If, for example, the coloured member 136 has a strong yellow colour, it will be immediately apparent to an operator when the valve is in its closed state, thereby clearly indicating that no attempts should be made to insert a catheter or a stent through the valve, whereas no yellow colour will be visible when the valve is in its open state, thereby clearly indicating that a catheter or a stent may be safely passed through the valve. It should be understood that the valve may alternatively be designed such that the coloured member is visible when the valve is in its open state and invisible in the closed state.

The coloured member 136 is shown in detail in FIG. 10, from which it is apparent that a projecting portion, such as preferably a barbed portion 144, allows the coloured member 136 to be connected to the proximal part 102 of the connector via a snap-lock made possible thanks to the barbed portion exhibiting a radial elasticity and substantially no longitudinal elasticity. The radial elasticity may be provided by longitudinally extending slits in the member 136, such slits being formed essentially like those slits 148 which are provided in the area of the barbed portion of the distal part 104 of the connector, see FIG. 16. As illustrated in FIG. 9, the proximal part 102 defines a recessed portion in the form of a collar 146, so that when the member 136 and the proximal part 102 are interconnected, the barbed portion firmly secures the member 136 in relation to the proximal part 102.

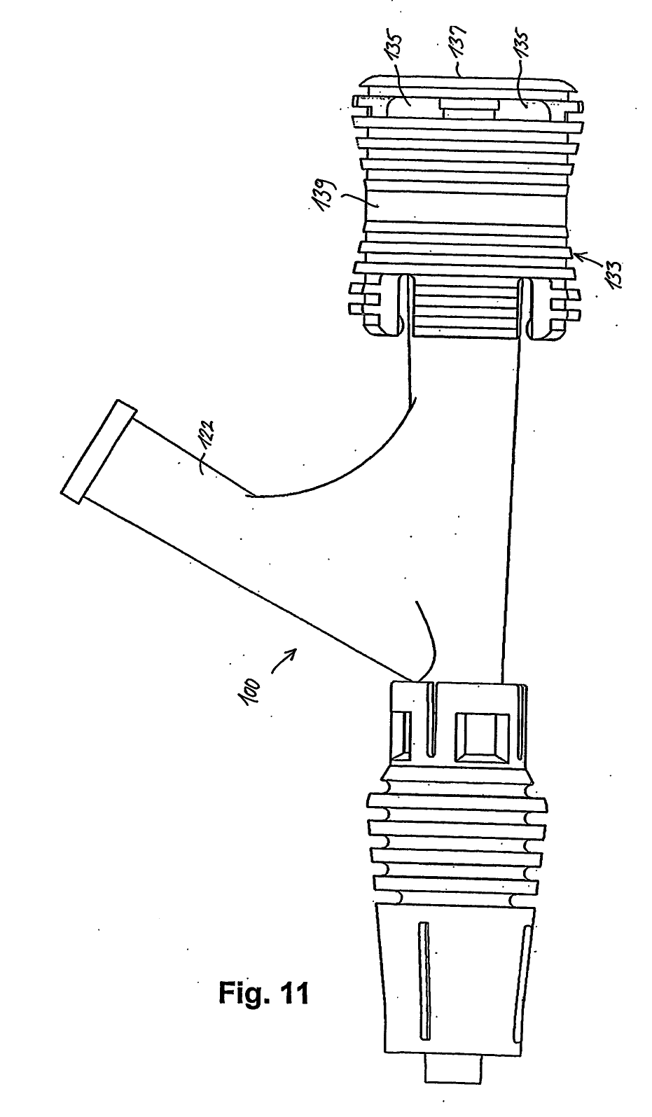

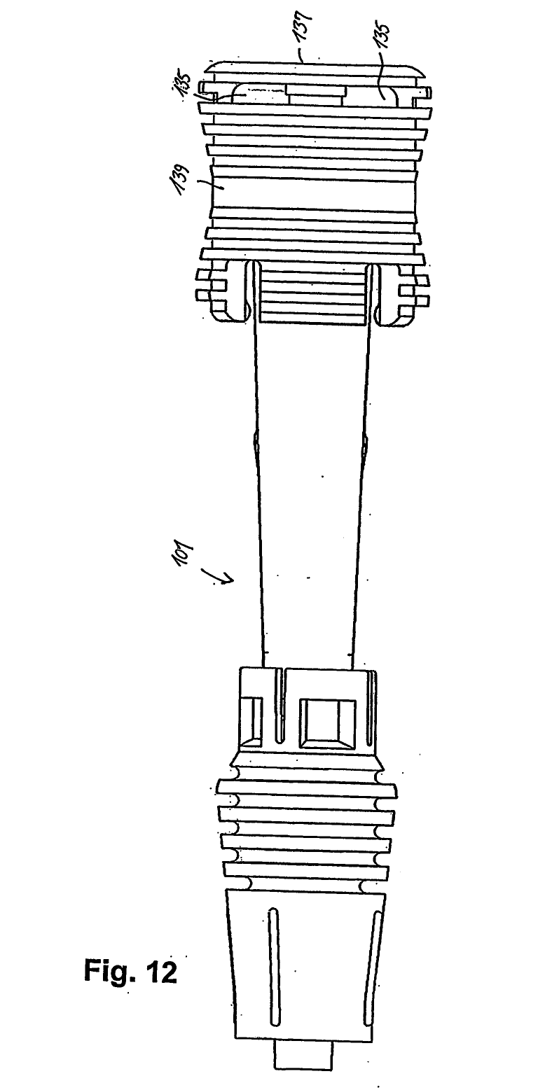

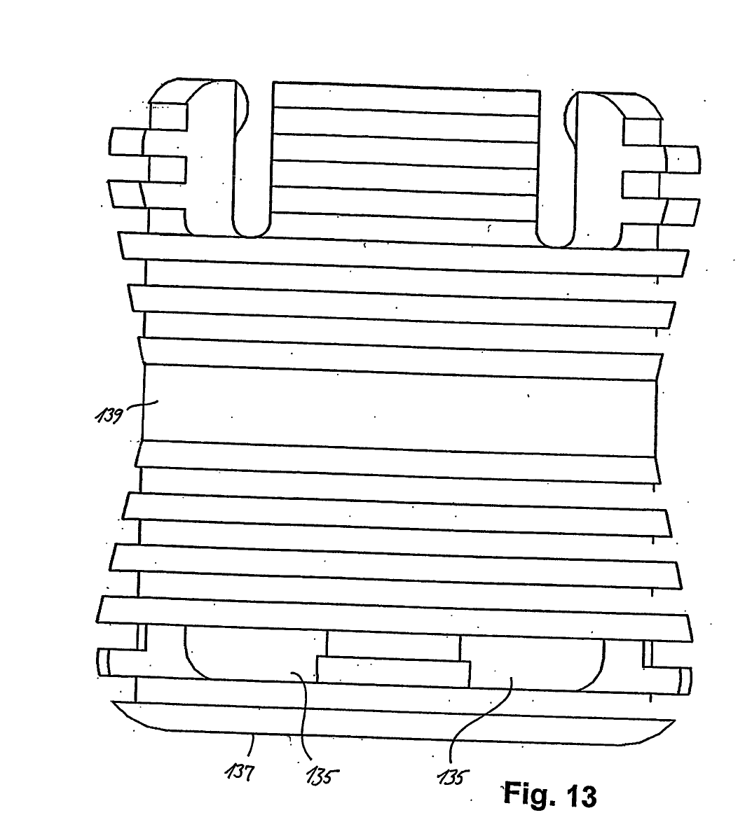

FIGS. 11-13 illustrate an alternative embodiment of a valve opener 133. Elements discussed above in connection with FIGS. 1-10 are referred to by the same reference numerals to the extent that like parts are referred to. It should, however, be understood that structural differences may exist, despite of the fact that the same reference numerals are employed. The connector 100 of FIGS. 11-13 comprises an opaque valve opener 133 with transparent or cut-out sections 135. A coloured member (not shown) similar or identical to the coloured member 136 described above in connection with FIGS. 2, 9 and 10 is provided. The coloured member is visible through the transparent or cut-out sections 135 when the valve opener is in its distal position, i.e. when the valve is open. In the closed state of the valve, i.e. when the valve opener 133 is in its proximal position, the coloured member is not visible. Preferably, the proximal end surface 137 is opaque, so that the coloured member is not visible through the end surface. In alternative embodiments, the sections 135 are also opaque, whereas a middle section 139 of the valve 133 may be transparent, so that the coloured member is visible in the open or in the closed state of the valve. In a yet further alternative embodiment, the sections 135 are transparent or cut-out, while also the middle section 139 is transparent. FIG. 12 illustrates a connector 101 without a side arm.

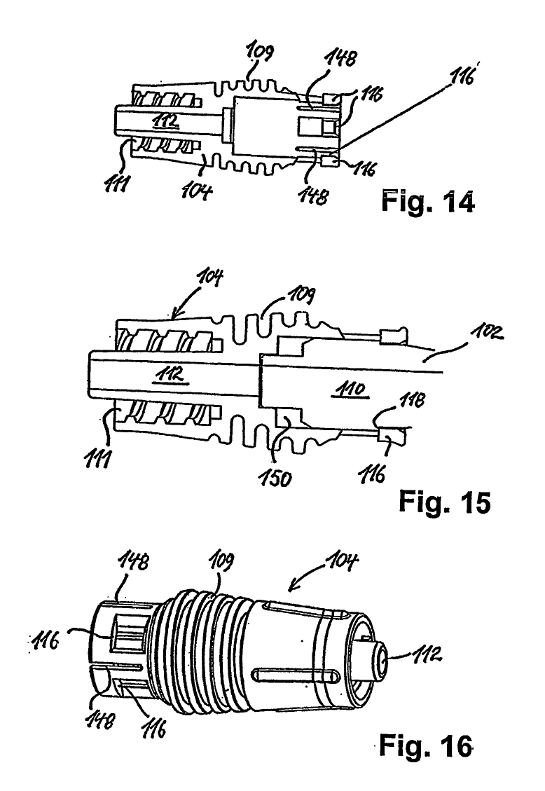

The interconnection between the distal and proximal parts 102 and 104, respectively, is based on the same principle as the interconnection between the coloured member 136 and the proximal part 102, as described above with reference to FIGS. 9 and 10. Thus, as illustrated in FIGS. 14 and 15, the proximal part 104 has a barbed portion 116 engaging a collar 118 of the proximal part 102. An annular space 150 is available for the O-ring which is not shown in FIGS. 14 and 15, but which is designated by reference numeral 120 in FIG. 1. The distal part 104 is shown in isolation in FIGS. 15 and 16, from which it is also apparent that a proximal end portion of the distal part comprises several barbed portions 116 arranged along the periphery of the distal part 104 and with slits 148 therebetween, the slits providing a radial flexibility which allows the second part 104 with the barbed portions 116 to engage the collar 118 of the proximal part 102 in a snap-locking manner.

The operation of the embodiment of the connector 100 described above with reference to the drawings is as follows:

1. A manifold (not shown) is attached to the side arm 122 of the connector.

2. The distal end of the connector is connected to a proximal end of a guiding catheter (not shown).

3. The connector is flushed with saline to remove air bobbles. Flushing of the valve 128 is achieved when the valve is in its open state.

4. A pressure/infusion device (not shown) is attached to the manifold. In order to avoid air aspiration, it should be assured that all connections are secure.

5. The guiding catheter is introduced, following a guiding catheter introduction procedure which is usually recommended by a manufacturer of the guiding catheter.

6. A guide wire (not shown), or a guide wire and a dilatation catheter (not shown) is/are introduced into the connector. A metal guide wire insertion tool (not shown) should be used when the guide wire is inserted alone to protect a top of the guide wire. A PTCA dilatation catheter can be inserted alone without opening the valve. However, the valve should be opened using the valve opener 132 for any device larger than a dilatation catheter, such as a stent, ultrasound catheter, etc.

7. Any procedure devised by the catheter or stent manufacturer is then followed.

The dimensions and other specifications of a preferred embodiment of the connector 100 are as follows:

| | |
|---|---|
| Inner diameter of narrowest portion: | 2.4 mm-9.0 mm. |
| Maximum diameter of device to be inserted: | 2.33 mm-8.0 mm. |
| Minimum diameter of device to be inserted: | 0.17 mm-1.10 mm. |
| Maximum pressure resistance with | 8 Atm or with Percutan |
| PTCA catheter and guide wire: | graft 1 atm. |
| Maxim pressure resistance without device: | 21 Atm-2 atm |
| Metallic insertion tool length: | 10 cm-2 cm |
| Metallic insertion tool inner diameter: | 0.64 mm-2.00 mm. |

The number of the interval mentioned first refers to PTCA and the second number of the interval refers to AAA graft (Percutaneous-Abdominal Aortic Aneurysm stent graft).

1. A closure for a valve (128) of a connector (100) of a haemostatic valve assembly, the closure comprising a closure member (130) which is made from a resilient material and which defines a first and a second, opposite end surface (156;160) and at least one passage slit (164), the passage slit being normally closed and extending between the two end surfaces, the passage slit being arranged to open by a tubular member (134) being extended therethrough, the passage slit (164) having a larger extent at the first surface than at the second surface.

2. A closure according to claim 1, comprising a plurality of passage slits (164) which define a first, common point of contact (166) on the first surface (156) and which extend radially outwardly from the point of contact (166) at the first surface (156).

3. A closure according to claim 2, wherein the plurality of passage slits (164) define a second, common point of contact (168) on the second surface (160).

4. A closure according to any of claims 1-3, wherein at least one of the passage slits (164) has a length on the second surface (160) which is at most 1/10th of the length of that passage slit on the first surface (156).

5. A closure according to claim 1, wherein at least a portion of the first end surface (156) and at least a portion of the second end surface (166) define two substantially parallel planes, and wherein an axis extending between the first and second common point of contact is substantially perpendicular to the two planes.

6. A closure according to claim 1, wherein at least a portion (170) of one of the first and second end surfaces (156;160) is concave.

7. A closure according to claim 6, wherein said concave portion (170) is provided on the second surface (160).

8. A closure according to claim 1, wherein a face (156) of the closure member (130) abuts a proximal end surface (152) of a main section (114) of the connector, one of said face and end surface (152;156) being provided with a protrusion (158) for engaging a corresponding indentation (154) provided in the other one of said face and said end surface (152;156).

9. A closure according to claim 10, wherein the closure member (130) is made from a resilient material which is adapted to deform in the area of said protrusion and said indentation when said face and said end surface (152;156) are biased towards each other, so as to thereby provide a liquid tight seal near an outer periphery of the passage (110;112) at a proximal end thereof.

10. A closure according to claim 8, wherein the protrusion (158) is integral with the closure member (130).

11. A connector (100) for a haemostatic valve assembly and comprising a closure according to claim 1.

12. A connector according to claim 11, wherein the valve (128) with the closure is arranged near a proximal end of the connector.

13. A connector according to claim 12, wherein the second surface (160) of the closure is oriented to face the proximal end of the connector.

14. A kit comprising a connector (100) according to claim 11, and a side arm tubing (126) for a side arm (122) of the connector.

15. A kit according to claim 14, further comprising a stopcock (124) to be connected to one end of the side arm tubing (126).
